(19) United States
(12) Patent Application Publication
(10) Pub. No.: US 2006/0251722 A1
(43) Pub. Date: Nov. 9, 2006

(54) Multi-component vitamin and mineral supplement for the optimal absorption of components

(75) Inventors: Stephen I. Bandak, Indianapolis, IN (US); Rahul Singhvi, Berwyn, PA (US)

Correspondence Address: PALMER & DODGE, LLP;KATHLEEN M. WILLIAMS, 111 HUNTINGTON AVENUE, BOSTON, MA, 02199, US

(73) Assignee: Novavax, Inc.

(21) Appl. No.: 11/120,767

(22) Filed: May 3, 2005

(51) Int. Cl. A61K 9/24

(52) U.S. Cl. 424/472

(57) ABSTRACT

The present invention relates to compositions and methods for administration of vitamin-mineral supplements, including those given to women prenatally. A multi-component vitamin delivery system is described comprising a first layer able to release a first set of components at a first location in the gastrointestinal tract, the first set of components includes iron in a form absorbable at the first location and a second layer able to release a second set of components at a second location in the gastrointestinal tract, the second location being further downstream in the gastrointestinal tract than the first location. The second set of components includes at least one material that reduces or inhibits the absorption of iron or whose absorption is inhibited by iron. Components are incorporated into the different layers of the delivery system to minimize the potential for interactions that may impair absorption and maximize conditions for optimal absorption.

### FIELD OF THE INVENTION

The present invention relates generally to multi-component vitamin and mineral supplements, and more particularly to multi-component vitamin and mineral delivery systems for administration to women prior to conception, throughout pregnancy, and post-pregnancy. The invention also includes methods of making and using such supplements.

### BACKGROUND OF THE INVENTION

An adequate intake of vitamins and minerals is important for health and can be obtained through a healthy diet. In the absence of such a diet, dietary supplements may be useful sources of one or more of these micronutrients that otherwise might be consumed in less than recommended amounts. Supplemental vitamins and minerals may also be required when gastrointestinal absorption is impaired, where there are excessive losses, or increased requirements. In particular, pregnancy increases micronutrient needs, and the consequences of deficiency of one or more vitamins or minerals can be serious for mother and child.

There are over 50 nutrients that are essential for good health when trying to conceive and during pregnancy. Three nutrients considered particularly important during pregnancy are iron, folic acid, and calcium.

Inadequate iron stores cause iron deficiency anemia. Iron deficiency in the early stages of gestation may increase the risk of low-birth weight or pre-term delivery. The need for iron increases during pregnancy and is even more important during the third trimester, when the fetus is storing iron for use after birth. Normally 10% of iron contained in foods is absorbed. During pregnancy, iron absorption increases to 20%. Due to the increased requirement during pregnancy, diet alone may not provide a sufficient source of iron. The health care professional often recommends an iron supplement to meet the fetus and mother's needs.

Calcium is required for normal growth and development of bones and teeth. Calcium also plays an important role in a variety of processes including muscle contraction and blood clotting. Vitamin D plays a critical role in the absorption of dietary calcium from the gastrointestinal tract. Because of the needs of the growing fetus, pregnancy depletes the mother's calcium supply. During pregnancy, calcium transfer from mother to developing fetus reaches an average of 270 mg per day by the third trimester. While drinking vitamin D fortified milk is an excellent source of both Vitamin D and calcium under normal circumstances, pregnant and lactating women often need supplemental vitamin D and calcium.

Folate, also called folic acid, is a B vitamin (vitamin B₉). It plays a crucial role in DNA and RNA synthesis and is critical to cell multiplication. Folate requirements are increased during the period of fetal growth and development characterized by rapid and widespread cell division. Folate deficiency results in birth defects known as neural tube defects (anencephaly and spina bifida). Folic acid supplementation during pregnancy is considered a routine part of pre-natal care.

Prenatal vitamin-mineral supplements as a class are distinguished by containing 1 mg or more of folic acid or folate and generally provide similar vitamin and mineral supplementation, useful in improving nutritional status prior to conception and throughout pregnancy. Ingredients of prenatal vitamins often include water-soluble vitamins (folic acid and vitamins C, B₁, B₂, B₃, B₆, and B₁₂), fat-soluble vitamins (A, D, E and K) and minerals and trace elements (most consistently calcium and iron but also frequently including zinc, chromium, iodine, magnesium, molybdenum, selenium and copper). Some prenatal supplements include a stool softener to counteract constipating side effects, primarily from unabsorbed iron.

The available vitamin and mineral supplements and those designed for prenatal vitamin-mineral supplementation generally differ in the specific type and amount of vitamins and minerals they contain. Little innovation has been applied to the formulation of these products. The changes in formulation development have been limited to extended release of iron to reduce side effects or minimize interactions, improved palatability (using chewable or liquid preparations with different flavors), and increasing the ease of swallowing (using different types of coatings).

U.S. Pat. No. 4,752,479 to Briggs et al., and U.S. Pat. No. 4,994, 283 to Mehansho et al. relate to delivery systems of vitamin-mineral supplements for increasing bioavailability of specific components. U.S. Pat. Nos. 5,494,678, 5,869,084, 6,228,388, 6,488,956 to Paradissis et al. focused on changing the composition of a supplement according to the stage of pregnancy or stage of life. U.S. Pat. No. 6,696,083 to Paradissis et al. focuses on continuous release of components over a 24 hour period for optimizing the repair or maintenance of nerve tissue occurring during sleep. However, despite the knowledge that has been accumulated regarding the absorption of, and interactions between, vitamins and minerals, no formulation has been developed that rationally applies this knowledge to multi-vitamin and mineral supplements.

For example, U.S. Pat. No. 4,752,479 to Briggs et al. attempts to minimize the potential for interactions involving iron. However, it suggests a mechanism to do so that delays the release of the iron and leads to suboptimal conditions for the absorption of the iron. U.S. Pat. No. 4,994,283 to Mehansho et al recognizes the potential for inhibition of iron absorption by calcium, but the suggested solution is a formulation requiring potentiating amounts of citrate, tartrate, ascorbate and/or fructose.

Thus, there is a need for a formulation of a vitamin-mineral supplement that takes advantage of known characteristics of gastrointestinal vitamin and mineral absorption, as described below.

It is well established that absorption of the different vitamins and minerals occurs at different sites in the alimentary tract. Iron is mainly absorbed in the duodenum and upper jejunum, where the acidity of the contents maintains iron in its soluble form^{4,5}. Beyond these sites, the enviromnent is more alkaline and iron is therefore less soluble. Drugs that reduce the acidity of the stomach, such as antacids, histamine-2 receptor antagonists, and proton pump inhibitors, may also interfere with iron absorption. Delayed release iron preparations are sometimes claimed to be better tolerated but may be less effective as iron is released beyond the site of optimal absorption. Despite the availability of a variety of delayed release iron preparations for several decades, authorities continue to preferentially recommend use of iron preparations with immediate release characteristics^{7,8}. For optimal absorption, supplements containing iron should be formulated to allow immediate release of the iron in the stomach.

It is also recognized that some components are important for the absorption of others. For example, vitamin C facilitates absorption of iron⁹. One mechanism for this enhanced absorption is the formation of an iron-ascorbic acid chelate in the stomach, which prevents the iron from binding with components of food (including phytates and tannins) known to inhibit its absorption^{10,11,12}. This has an impact not only on the amount of iron made available for metabolic processes, but also on the side effects of the supplement. The presence of unabsorbed iron in the lumen of the gut may cause constipation. Pregnant women are pre-disposed to constipation and multi-vitamin and mineral supplements are known to be capable of making the problem worse. Optimizing iron absorption is important therefore with respect to both efficacy and tolerance. A formulation designed to maximize absorption of iron requires that the iron and the vitamin C be released simultaneously in the upper gastrointestinal tract.

A critical aspect of this invention is the separation of components that may interfere with the optimal absorption of others. Calcium interferes with the absorption of iron¹⁵. Any of the most commonly used forms of calcium supplement (calcium carbonate, citrate and phosphate) inhibit absorption of supplemental iron when both are taken with food, while calcium phosphate and citrate will also do so even when given with iron in the fasting state¹⁶. Separating the time of ingestion of the calcium and the iron or formulating a supplement to allow separation of the sites of dissolution entirely avoids inhibition of iron absorption by calcium¹⁷. Optimal absorption of iron from a supplement that also contains calcium therefore requires separation of the dissolution of the iron and the calcium from the supplement, with the release of the calcium taking place a significant interval after that of the iron.

A second significant instance of micronutrient interaction is the inhibition of zinc absorption caused by iron^{14,18,19}. As the mechanism appears to be competition for a common binding site on the mucosal surface responsible for uptake of both elements from the gut²⁰, separation of the dissolution of the iron from that of the zinc minimizes the potential for this interaction. An aspect of this invention is that in order to optimize absorption of zinc from a supplement that also contains iron, a formulation is proposed that would delay the dissolution of zinc beyond the location of iron release.

Before vitamin B₁₂ can be absorbed and used by the body, binding must occur with intrinsic factor, secreted by gastric cells. Receptors on the villi of the distal small intestine take up the intrinsic factor/B₁₂ complex only in the presence of calcium. A supplement formulated to maximize the absorption of vitamin B₁₂ would allow its dissolution in the stomach to maximize the binding to intrinsic factor, and would release calcium distally in the small intestine to optimize uptake of the intrinsic factor/B₁₂ complex. Both factors are aspects of this invention.

Components of food (phytates, oxalates and tannins) may bind to mineral components of supplements (including calcium, iron and zinc) and reduce their availability for absorption. An important aspect of this invention is that the dissolution of components is designed to minimize the time available for such binding to take place by releasing them at a location optimal for their absorption.

The fat-soluble vitamins are co-absorbed in the small intestine at the same time as fat itself. Fat absorption takes place only after bile and pancreatic lipase mix with the contents of the duodenum changing dietary fat into micelles that are absorbed by the brush border of the small intestine. This invention contemplates delaying the release of fat-soluble vitamins from the supplement until after the formation of micelles in order to optimize conditions for their incorporation into the micelles and subsequent absorption.

The issue of interactions is particularly relevant at the concentrations of vitamins and minerals administered in supplements, which are higher than dietary concentrations, and has important implications for a multiple micronutrient supplement regimen in pregnancy. While some of these issues can be ameliorated or avoided entirely by taking individual vitamins and minerals at different times, there are clearly limitations to the number of tablets women can be expected to take regularly without impacting compliance. Accordingly, a need in the art remains for a vitamin and mineral supplement that is intended to optimize absorption, minimize gastrointestinal side effects, and be practical for daily use.

### SUMMARY OF THE INVENTION

The present invention provides a multi-component vitamin delivery system that is particularly useful in promoting the health of pre- and postnatal mothers and their babies through pregnancy and lactation. The multi-component vitamin delivery system is designed to optimize absorption of the different vitamins and minerals.

The multi-component system for delivering a vitamin to a subject, such as a human, including a first layer adapted to release a first component at a first location of the gastrointestinal (GI) tract and a second layer adapted to release a second component further downstream in the GI tract. The first and second components are placed in separate layers to optimize the absorption of the components and to minimize detrimental interactions between the first and second components. At least one of the first and second components includes a vitamin.

The first component can include at least one vitamin, a mineral and/or a trace element that is to be released to the first location of the GI tract. A preferred mineral in the first layer is iron, preferably in a form absorbable at the first location. Preferred materials for inclusion in the first layer include water-soluble vitamins selected from the group consisting of vitamin C and the B-complex vitamins, particularly folic acid and vitamin B₁₂.

The second component can include at least one vitamin, mineral or trace element that is to be released at the second location within the GI tract. Preferred minerals for the second component are calcium, chromium, iodine, zinc, magnesium, molybdenum, selenium, copper or a salt or a mixture thereof. The preferred minerals in the second layer are calcium and zinc. The vitamin in the second component can be a fat-soluble vitamin, such as vitamin A, vitamin D, vitamin E, vitamin K or a mixture thereof. The second location is further downstream in said gastrointestinal tract than said first location; that is, the materials from the second layer are released at a time and location separated from that of the materials from the first layer.

If iron is included in the first layer, the second set of components will include any material that is to be included in the delivery system but hinders the absorption of iron or whose absorption is hindered by iron. These include calcium, zinc, and salts and mixtures thereof. The second set of components may include one or more minerals and trace elements selected from the group consisting of chromium, calcium, iodine, zinc, magnesium, molybdenum, selenium and copper.

The first location is normally selected from the group consisting of the stomach and the duodenum. In contrast, the second location is selected from the group consisting of the small and large intestines.

The multi-component vitamin delivery system can be in a variety of forms. Due to the first layer having to deliver its material first, it is normally exposed or covered with an easily dissolvable coating. Preferred coating layers for the first layer of the delivery system are selected from the group consisting of sugar coatings, film coatings and enteric coatings. There also may be a second coating layer that allows release of the second set of components from said second layer at the second location. This second coating layer can be selected from the group consisting of a microporous membrane and an enteric coating.

The second layer of the delivery system may be partially or totally surrounded by the first layer. In one format, a bi- or multi-layer tablet is formed with each layer being adjacent to the other like layers on a cake. Alternatively, a tablet or caplet is used and the first layer completely surrounds the second layer (and any additional layers). The layers may also be shaped to provide optimum release of the components.

A preferred dosage form for providing the vitamins and/or minerals of the delivery system to a subject is an oral dosage form. The delivery system is selected so that various types of release characteristics, including immediate release, extended release, pulsed release, delayed release, timed release, variable release, controlled release and combinations thereof may be utilized. The oral dosage form can be a tablet or caplet.

The invention also provides a method of making the multi-component vitamin delivery system previously described. The method includes mixing the first set of components that are to be released at a first location in the gastrointestinal tract together to form a first layer. Similarly, the second set of components that are to be released at a second location in the gastrointestinal tract are mixed separately from the first. The second set of components are kept separate from the first set of components because the second set is to be released at a second location further downstream in said gastrointestinal tract than said first location. After the first and second sets of components are prepared, they are formed into the first and second layers, respectively, of the delivery system. The first and second layers are then combined, while maintaining their individual integrities, to form said delivery system.

The method may include the steps of coating said second layer with a second coating layer through which the components of the second layer can diffuse or the second coating layer can dissolve. The second coating layer provides a lag-time between delivery of components from the first layer and the subsequent delivery of components from the second layer. Similarly, the first layer may be coated with a first coating layer that delays the release of the components of said first layer. The first coating layer may be placed on the first layer by a process selected from spray coating and compression coating.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIGS. 1A-1E illustrate five alternative bi-layer tablets wherein the first layer partially, but not completely, covers the second layer, and various coatings are used.

FIGS. 2A-2E illustrate five alternative bi-layer tablets according to an embodiment of the invention wherein the first layer completely surrounds the second layer and various coatings are used.

### DETAILED DESCRIPTION

The following definitions are used to describe the various aspects and characteristics of the invention.

The term “immediate release” means that the release of the material from the tablet is not delayed once at its intended location, e.g., the rapid break-up and delivery of a portion of the tablet in the upper gastrointestinal tract.

The term “modified release” is intended to exclude immediate release and to encompass extended release, pulsed release, controlled release, sustained release, delayed release, timed release, variable release and combinations thereof.

The term “time delay” denotes the duration of time between administration of the composition of the present invention and the release of the components from a particular layer.

The term “lag time” denotes the time between delivery of components from one layer and the subsequent delivery of components from another layer.

The term “prenatally relevant amounts” means that the amounts meet the currently accepted recommended dietary allowances and intakes for women prior to conception, throughout pregnancy, and post-pregnancy.

The term “modified release matrix material” means materials that are capable of modifying the release of an active ingredient dispersed therein in vitro or in vivo, and include materials like hydrophilic polymers, hydrophobic polymers and mixtures thereof.

The present invention provides a multi-component delivery system that is useful for administering varying dosage levels of vitamins, minerals and/or supplements to a subject, such as a human. The vitamin delivery system of the present invention is especially useful for administering vitamins, minerals and supplements to nonpregnant women and women at various stages of pregnancy and post-pregnancy including lactating women.

The delivery system of the invention has a first layer that is able to release a first set of components at a first location in the gastrointestinal tract, and preferably includes iron in a form absorbable at the first location. The first layer may also include at least one water-soluble vitamin, mineral and trace element that is to be released at the first location. The first location is selected from the group consisting of the stomach and the duodenum.

The delivery system also has a second layer able to release a second set of components at a second location in the gastrointestinal tract, the second location being further downstream in the gastrointestinal tract than the first location. The second set of components includes at least one material that hinders the absorption of iron or whose absorption is inhibited or reduced by iron and is selected from the group consisting of calcium, zinc, and salts and mixtures thereof. The second set of components may also include at least one fat-soluble vitamin, mineral or trace element that is to be released at the second location. The second location is selected from the group consisting of the small and large intestines.

One advantage of separating the components into layers is that it allows control of the release from the layers at a time and location that corresponds to the location of optimal absorption in the gastrointestinal tract. This separation of components limits the potential for unwanted binding to food and other particles that may reduce the bioavailability of the components. By separating various components in the different layers of the composition, they can aid each other to optimize absorption, and keep the interference of components in the gastrointestinal tract minimized.

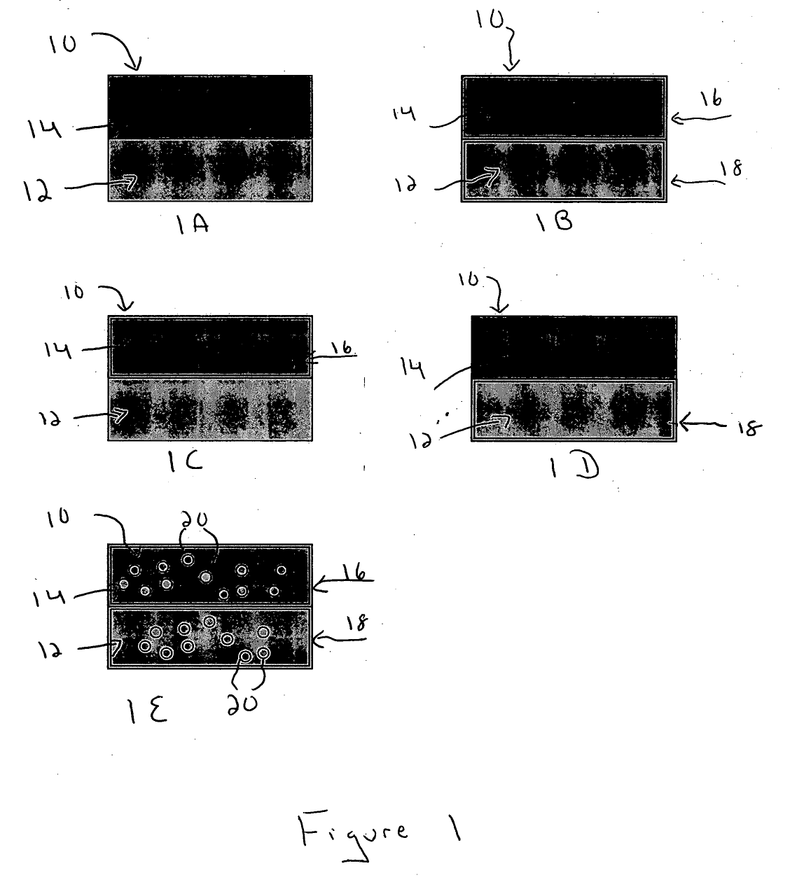

FIGS. 1A-1E show a schematic of five alternative bi-layer tablets in a layer cake-like format according to the present invention. In FIG. 1A, the composition is a bi-layer tablet 10 that includes a first layer 14 containing prenatally relevant amounts of water-soluble vitamins, minerals and trace elements. Second layer 12 is partially surrounded by the first layer 14 and partially open and containing prenatally relevant amounts of fat-soluble vitamins, minerals and trace elements. As shown in FIGS. 1B-1D, the first layer 14 may be optionally surrounded by a cover layer 16. Cover layer 16 may include, but is not limited to, a sugar coating (for color and to cover bad taste), a film coating (which can be a polymeric material used to color and hide taste), and an enteric coating (which acts as a delayed-action layer that erodes in differently acidic or alkaline environments of the gastrointestinal tract). Optionally, a coating layer 18 may surround second layer 12. Coating layer 18, may include, but is not limited to, a microporous coating (for diffusion of components through micropores); and a coating which acts to delay the time between delivery of components from the first layer 14 and the subsequent delivery of components from the second layer 12. FIG. 1E shows a composition of the present invention where the components themselves are optionally particulates and the particulates are individually surrounded by a coating layer 20.

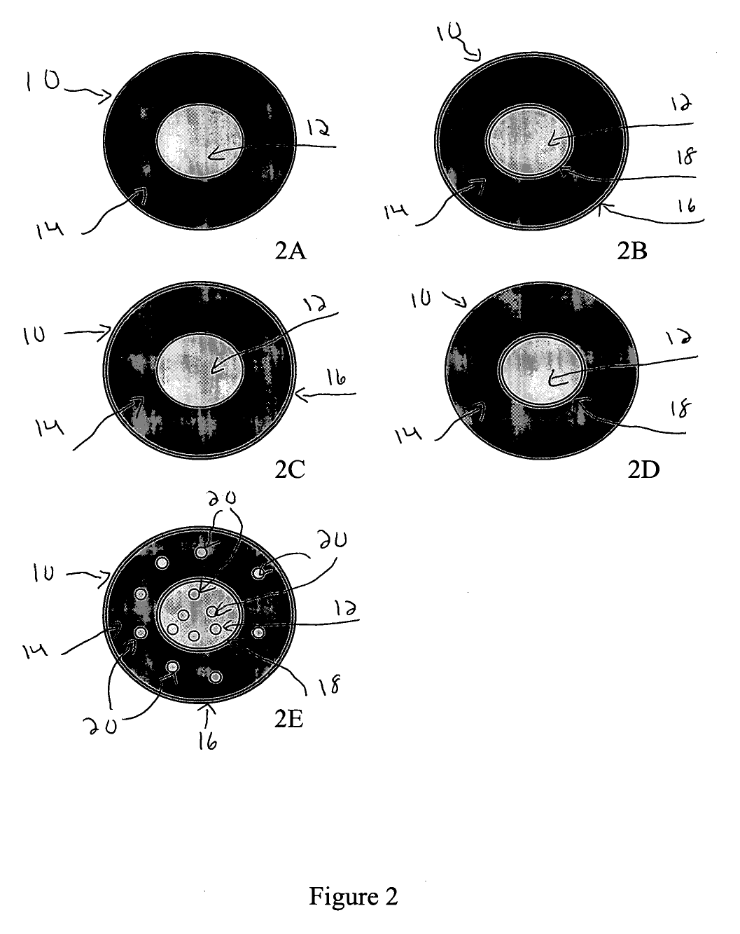

FIGS. 2A-2E show a schematic of five alternative bi-layer tablets wherein one layer surround the other according to a different embodiment of the invention. In FIG. 2A, the composition is a bi-layer tablet 10 that includes a first layer 14 containing prenatally relevant amounts of iron, water-soluble vitamins, minerals and trace elements. Tablet 10 also includes a second layer 12, completely surrounded by first layer 14, containing prenatally relevant amounts of fat-soluble vitamins, minerals and trace elements. As shown in FIGS. 2B-2D, the first layer 14 may be optionally surrounded by a cover layer 16 such as a sugar coating (for color and to cover bad taste); or a film coating (which can be a polymeric material as used to color and hide taste). Also, optionally, the second layer 12 may be surrounded by a coating layer 18 that delays release until a given location in the gastrointestinal tract (e.g., an enteric coating which delays the release until the intestine). Coating layer 18 may be a microporous coating (for diffusion of components through micropores) or a coating which acts to delay the time between delivery of components from the first layer 14 and the subsequent delivery of components from the second layer 12. FIG. 2E shows a composition of the present invention where the components themselves are optionally particulates and the particulates are individually surrounded by a coating layer 20.

The first layer 14 of tablet 10 includes iron in an absorbable form and may include water-soluble components. Possible water-soluble components include, but are not limited to, vitamin C and the B-vitamins (for example, thiamin (B₁), riboflavin (B₂), niacin (B₃), folic acid (B₉), pyridoxine (B₆) and cyanocobalamin (B₁₂)). The components of first layer 14 are selected to include materials that are to be released in the upper portion of the gastrointestinal tract. The choice of components for the first layer 14 is determined based on optimal absorption in the gastrointestinal tract, reduction in the negative interference effects between components, and minimizing the side effects of the various components. For example, intrinsic factor, which is secreted by the cells lining the stomach, is necessary for the absorption of vitamin B₁₂ Thus, it is ideal for vitamin B₁₂ to be present in the first layer 14 where it will be released in the upper gastrointestinal tract so that it can bind with intrinsic factor. The absorption of iron occurs predominantly in the upper gastrointestinal tract, so placement of iron in the first layer 14 is required.

As noted, first layer 14 of bi-layer tablet 10 may include water-soluble vitamins, minerals and trace elements selected from the group consisting of vitamin C and its derivatives, the B-complex vitamins and their derivatives and iron and its derivatives. Because vitamin C is one of the most potent enhancers of iron absorption, it is desirable to place the vitamin C with the iron in the first layer 14 of bi-layer tablet 10.

Vitamin C derivatives are any compounds or combinations of compounds having vitamin C or antiscorbutic activity. Vitamin C derivatives include salts of ascorbic acid, alkaline salts of ascorbic acid, esters of ascorbic acid, oxidation products of ascorbic acid, vitamin C precursors, metabolites of ascorbic acid and its derivatives and combinations thereof. Salts of ascorbic acid useful in the present invention include mineral and multi-mineral ascorbates. Metabolites of ascorbic acid and its derivatives include aldonic acids, aldono-lactones, aldono-lactides, edible salts of aldonic acids and mixtures thereof.

Non-limiting exemplary vitamin C derivatives include magnesium ascorbate, potassium ascorbate, sodium ascorbate, dehydroascorbic acid, L-ascorbic acid 2-0-sulfate, L-ascorbic acid 2-0-phosphate, L-ascorbic acid 3-0-phosphate, L-ascorbic acid 6-hexadecanoate, L-ascorbic acid monostearate, L-ascorbic acid dipalmitate, L-threonic acid, L-xylonic acid, L-lyxonic acid and combinations thereof. While calcium ascorbate and zinc ascorbate are often used in multi-vitamins, care should be used in including these because of the potential interactions between iron and calcium and zinc described above. Preferably, the Vitamin C derivative is magnesium ascorbate, potassium ascorbate, sodium ascorbate and combinations thereof. The Recommended Dietary Allowance (RDA) of vitamin C for non-pregnant women is about 60 mg, during pregnancy it is about 70 mg, and during lactation it increases to about 95 mg.

Non-limiting exemplary iron compounds include ferrous fumarate, ferrous sulfate, ferric chloride, ferrous gluconate, ferrous lactate, ferrous tartrate, iron-sugar-carboxylate complexes, ferrous succinate, ferrous glutamate, ferrous citrate, ferrous pyrophosphate, ferrous cholinisocitrate, ferrous carbonate, carbonyl iron and mixtures thereof. Preferably, the iron compound is ferrous fumarate, carbonyl iron or mixtures thereof. The large surface area of carbonyl iron particles results in higher reactivity of carbonyl iron particles with gastric acid in the stomach and consequently higher absorption rates. Thus, carbonyl iron exhibits 2-5 times higher bioavailability than other elemental iron forms²⁰. Within the stomach, carbonyl iron is oxidized to the ferrous form of iron using naturally produced stomach acids. This provides a delayed-release mechanism regulated by the body's own acid secretion. Due to its natural, self-regulated delayed release, carbonyl iron is significantly safer than other ferrous iron salts used in ordinary prenatal vitamins. The RDA of iron for women is normally about 15 mg, 3.0 mg of which is absorbed; and during pregnancy, while it is also about 15 mg, anemia is often a problem and supplements of 30-60 mg are recommended.

Vitamin B₁ plays a role in carbohydrate metabolism and neural function and is available in forms known to those of skill in the art, including the form of thiamin mononitrate. The RDA of vitamin B₁ for a non-pregnant woman is normally about 1.1 mg, during pregnancy it is about 1.5, and during lactation it is about 1.6 mg.

Riboflavin (a vitamin B₂ derivative) is a component of two flavin coenzymes, flavin mononucleotide (FMN) and flavin adenine dinucleotide (FAD). Flavoenzymes also play a role in a number of metabolic pathways such as amino acid deamination, purine degradation, and fatty acid oxidation and thus help to maintain carbohydrate, amino acid, and lipid metabolism. The RDA of vitamin B₂ for women is normally about 1.3 mg and during pregnancy and lactation it is about 1.5 mg.

Niacin, also called vitamin B₃, is the common name for two compounds: nicotinic acid (also called niacin) and niacinamide (also called nicotinamide). Niacin is particularly important for maintaining healthy levels and types of fatty acids. Niacin is also required for the synthesis of pyroxidine, riboflavin, and folic acid. Nicotinamide adenine dinucleotide (NAD) and NAD phosphate (NADP) are active coenzymes of niacin. These coenzymes are involved in numerous enzymatic reactions such as glycolysis, fatty acid metabolism, and steroid synthesis²¹. The RDA of niacin for women is normally 15 mg, and during pregnancy and lactation it increases to about 20 mg.

The administration of pyridoxine (a vitamin B₆) may reduce the levels of homocysteine²² The active forms of pyridoxine, pyridoxal-5′-phosphate (PLP) and pyridoxamine-5′-phosphate are coenzymes for numerous enzymes and as such, are important for gluconeogenesis, niacin formation, and erythrocyte metabolism. Pyridoxine is a coenzyme for both cystathionine synthase and cystathionase, enzymes that catalyze the formation of cysteine from methionine. Homocysteine is an intermediate in this process and elevated levels of plasma homocysteine are recognized as a risk factor for both vascular disease²³ and neural tube defects²⁴. Vitamin B₆ is available in forms known to those of skill in the art, including the form of pyridoxine hydrochloride. The RDA of vitamin B₆ for a non-pregnant woman is normally 1.6 mg, during pregancy it is 2.2 mg, and during lactation is about 2.1 mg.

The B-complex vitamin folic acid (vitamin B₉) has demonstrated the ability to prevent neural tube defects such as spina bifida caused by disturbed homocysteine metabolism24,25,26,27. Further, folic acid is important for the formation of red and white blood cells within bone marrow and plays a role in heme formation. The RDA of folic acid for women is normally 180 μg, during pregnancy is about 250 μg, and during lactation it is about 280 μg.

Cobalamin (a form of vitamin B₁₂) can be converted to the active coenzymes: methylcobalamin and 5′-deoxyadenosylcobalamin. These coenzymes are necessary for folic acid metabolism, conversion of coenzyme A, and myelin synthesis. For example, methylcobalamin catalyzes the demethylation of a folate cofactor that is involved in DNA synthesis. A lack of demethylation may result in folic acid deficiency. Deoxyadenosylcobalamin is the coenzyme for the conversion of methylmalonyl-CoA to succinyl-CoA, which plays a role in the citric acid cycle. Importantly, cobalamin, along with pyridoxine and folic acid in implicated in the proper metabolism of homocysteine. Cobalamin is available as cyanocobalamin, methylcobalamin, hydroxocobalamin, adenosylcobalamin, and hydroxycyanocobalamin. The RDA of vitamin B₁₂ for women is normally about 2.0 μg, during pregnancy it is about 2.2 μg, and during lactation it increases to about 2.6 μg.

The second layer 12 of tablet 10 may include one or more fat-soluble components, including, but not limited to, vitamins A, E, D and K. In addition, one or more minerals and trace elements, including, but not limited to chromium, calcium, zinc, magnesium, molybdenum, selenium, copper and iodine, may be included in second layer 12. The components of the second layer 12 will be released after the components of the first layer 14, and at a point downstream from the release of the components of the first layer 14. It is well documented that calcium interferes with the absorption of iron, therefore the calcium and iron should be provided in different layers. It has also been shown that iron interferes with the absorption of zinc, so the iron and zinc are provided in different layers. Because the site of zinc absorption is distal to the site for iron, and these two components would interfere with one another, the zinc should be present in second layer 12 as well as the calcium. It is also well known that the fat-soluble vitamins, namely vitamins A, E, D and K, are co-absorbed with fat in the lower small intestine, so they should be provided in the second layer 12.

Vitamin D used in the present compositions can include any of the forms of vitamin D or any of the precursors of 1,25-dihydroxycholecalciferol. The RDA of vitamin D for women is normally 10 μg; during pregnancy it stays at about 10 μg and during lactation is about 12 μg.

Vitamin A is important for maintenance of visual function. Its main influence is on the retina, but it also aids glycoprotein synthesis and promotes cellular growth and differentiation in other tissues. Vitamin A can be provided, for example, in the form of beta-carotene, retinol equivalents, vitamin A acetate, and vitamin A palmitate. The RDA of vitamin A is normally 800 μg, during pregnancy it is about 800 μg, and during lactation it increases to about 1300 μg.

Calcium-based compounds include, but are not limited to, any of the well known calcium supplements, such as calcium carbonate, calcium sulfate, calcium oxide, calcium hydroxide, calcium apatite, calcium citrate-malate, bone meal, oyster shell, calcium gluconate, calcium lactate, calcium phosphate, calcium levulinate, and the like. Derivatives of calcium compounds include salts of calcium, alkaline salts of calcium, esters of calcium, and combinations thereof. The salts and alkaline salts refer to those regularly used organic or inorganic salts that are acceptable for pharmaceutical use. The calcium of the present composition may be from any source. The RDA of calcium for women is normally 1200 mg, and during pregnancy and lactation it is also about 1200 mg.

Vitamin E can be present as α-, β-, γ-, or δ-tocopherol or as a mixture, or as an isomer thereof, such as D-α-tocopherol acetate or DL-α-tocopherol acetate. Salts of vitamin E include, but are not limited to, an acetate, or acid succinate salt. The RDA of vitamin E is normally about 8 mg, during pregnancy it is about 10 mg, and during lactation it increases to about 12 mg.

Vitamin K is required for synthesis of clotting factors VII, IX, and X. Transportation of vitamin K from mother to fetus is limited; nevertheless, significant bleeding problems in the fetus are rare. However, newborn infants are often functionally deficient in vitamin K and require parenteral supplementation at birth. The RDA of vitamin K for a woman is normally about 55 mg, and during pregnancy and lactation it is about 65 mg.

Elemental magnesium derivatives, such as magnesium stearate, magnesium carbonate, magnesium oxide, magnesium hydroxide, magnesium sulfate and combinations thereof may be provided in the compositions of the invention. Preferably, the magnesium compound is magnesium stearate, magnesium oxide or a combination thereof. The magnesium may be present in any appropriate amount but preferably is present in an amount ranging from about 10 mg to about 500 mg. The RDA of magnesium for a woman is normally about 280 mg, and during pregnancy and lactation it increases to about 355 mg.

Pharmaceutically acceptable copper compounds include cupric oxide, cupric sulfate, cupric gluconate and combinations thereof, with cupric oxide being preferred. Preferably, the copper compound is present in the compositions in an amount ranging from about 0.1 mg to about 10 mg.

Useful pharmaceutically acceptable zinc compounds for inclusion in the present compositions include, without limitation, zinc sulfate, zinc chloride, zinc oxide and combinations thereof. Preferably, the compound is zinc oxide. The RDA of zinc for a woman is normally 12 mg; during pregnancy and lactation it increases to about 19 mg.

Optionally, the compositions of the present invention may further contain a laxative such as docusate sodium in an amount ranging from about 25-75 mg, preferably an amount of about 40-60 mg, and most preferably an amount of about 50 mg/tablet. Other forms may include docusate calcium.

The compositions of the present invention may have a cover layer 16 applied to the first layer 14. Cover layer 14 can cause delay in the release of components from the first layer 14 after administration or have other uses. For example, cover layer 14 may have a colorant to improve the tablet appearance or have a flavoring to mask the bad taste of the composition (e.g., sugar or film coating). Further, a coating layer 18 may be applied to the second layer 12. Coating layer 18 may cause a lag time between the release of components from the first layer 14 and the release of components from the second layer 12 and may change the location of delivery (e.g., an enteric coating which delays the release until the intestine). Altering the composition of coating layer 18 and/or the amount of the coating utilized may vary the duration of the lag time. Thus, the duration of the lag time can be designed to mimic a desired plasma profile. Further, the individual components present in the first 14 and second 12 layers may be in particulate form and may be surrounded by a coating layer 20.

While the multi-component delivery system of the present invention may be provided in any suitable dosage form known in the art, preferably the multi-component delivery system is in an oral dosage form. The oral dosage form may be selected from the group consisting of tablets and caplets. In a preferred embodiment, the delivery system is in the form of a tablet. More specifically, multi-layer tablet forms are preferred. Even more specifically, bi-layer tablet forms are preferred. The geometry of the first and second layer may be selected to provide optimum delivery parameters as well. This may include shaping one or both of the layers to provide steady state or controlled release of materials as desired. Those skilled in the art will recognize that the multi-component delivery system of the present invention may be a multi-layer tablet comprising two, three, four and five layers, with various components in each layer.

It is also possible for the dosage form to combine various forms of release, which include, without limitation, immediate release, extended release, pulse release, variable release, controlled release, timed release, sustained release, delayed release, long acting, and combinations thereon. The ability to obtain immediate release, extended release, pulse release, variable release, controlled release, timed release, sustained release, delayed release, long acting characteristics and combinations thereof is performed using well known procedures and technique.

The time release characteristics for the release of the components from each of the layers may be varied by modifying the composition of each layer, including modifying any of the excipients or coatings which may be present. For example, changing the composition or amount of the covering or coating layers may be used to control the release of the components. Similarly, when modified release is facilitated by the inclusion of a specific matrix material, controlling the choice and amount of modified release matrix material utilized may be used to control the release of the active components. The matrix material may be present, either surrounding individual components of the composition or over a layer of the composition, in any amount that is sufficient to yield the desired time release or time lag between release of the components.

Any coating material that modifies the release of the components in the desired manner may be used. In particular, coating materials suitable for use in the practice of the invention include, but are not limited to polymer coating materials, hydrogels and gel-forming materials and hydrophilic polymers. Preferred polymer coating materials are cellulose acetate phthalate, cellulose acetate trimaletate, hydroxypropyl methylcellulose phthalate, polyvinyl acetate phthalate, amino methacrylate copolymers such as Eudragit® RS and RL, polyacrylic acid and polyacrylate and methacrylate copolymers such as Eudragit® S and L, polyvinyl acetaldiethylamino acetate, hydroxypropyl methylcellulose acetate succinate, and shellac.

Preferred hydrogels and gel-forming materials include carboxyvinyl polymers, sodium alginate, sodium carmellose, calcium carmellose, sodium carboxymethyl starch, polyvinyl alcohol, hydroxyethyl cellulose, methyl cellulose, gelatin, starch, and cellulose based cross-linked polymers in which the degree of crosslinking is low so as to facilitate adsorption of water and expansion of the polymer matrix, hydroxypropyl cellulose, hydroxypropyl methylcellulose, crosslinked starch, microcrystalline cellulose, chitin, aminoacryl-methacrylate copolymer (Eudragit® RS-PM), pullulan, collagen, casein, agar, gum arabic, sodium carboxymethyl cellulose, (swellable hydrophilic polymers) poly(hydroxyalkyl methacrylate) (molecular weight about 5 k-5,000 k), polyvinylpyrrolidone (molecular weight about 10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene pectin (molecular weight about 30 k-300 k), polysaccharides such as agar, acacia, karaya, tragacanth, algins and guar, polyacrylamides, Polyox® polyethylene oxides (molecular weight about 100 k-5,000 k), AquaKeep® acrylate polymers, diesters of polyglucan, crosslinked polyvinyl alcohol and poly N-vinyl-2-pyrrolidone, and sodium starch glycolate (e.g., Explotab®).

Preferred hydrophilic polymers include polysaccharides, methyl cellulose, sodium or calcium carboxymethyl cellulose, hydroxypropyl methyl cellulose, hydroxypropyl cellulose, hydroxyethyl cellulose, nitrocellulose, carboxymethyl cellulose, cellulose ethers, polyethylene oxides (e.g. Polyox®, Union Carbide), methylethyl cellulose, ethylhydroxy ethylcellulose, cellulose acetate, cellulose butyrate, cellulose propionate, gelatin, collagen, starch, maltodextrin, pullulan, polyvinylpyrrolidone, polyvinyl alcohol, polyvinyl acetate, glycerol fatty acid esters, polyacrylamide, polyacrylic acid, copolymers of methacrylic acid or methacrylic acid (e.g., Eudragit®), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof.

As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, glycerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

If coating layer 18 may be any suitable modified release matrix material or suitable combination of modified release matrix materials. Such materials are known to those skilled in the art. Modified release matrix materials suitable for the practice of the present invention include, but are not limited to, microcrystalline cellulose, sodium carboxymethylcellulose, hydroxyalkylcelluloses (e.g., hydroxypropylmethylcellulose and hydroxypropylcellulose), polyethylene oxide, alkylcelluloses (e.g., methylcellulose and ethylcellulose), polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate and mixtures thereof.

The compositions of the invention release are selected such that substantially all of the components contained in first layer 14 are released prior to release of the components from second layer 12. Release of the components from second layer 12 may be delayed as detailed above by the use of a modified release coating layer and/or a modified release matrix material.

It will be understood that the components and amounts of the components within each layer of the multi-layer tablet can be altered as desired. Alteration of the components may be in response to the needs of an individual, the stage of pregnancy (i.e., first, second or third trimester), or whether the individual is lactating or non-lactating.

Table 1 lists the recommended dietary allowances and intakes for pregnant women and women who are lactating (i.e., nursing) for some of the mineral and vitamin components of the present delivery system. Note that these values are approximate recommended dietary allowances and that “prenatally relevant amounts” of components may change over time.

TABLE 1
Recommended Dietary Allowances

| Nutrient | Pregnant Women | | Lactating Women | |
|---|---|---|---|---|
| Vitamin A | 800 | μg | 1300 | μg |
| Vitamin B₁ | 1.5 | mg | 1.6 | mg |
| Vitamin B₂ | 1.5 | mg | 1.5 | mg |
| Niacin (Vitamin B₃) | 20 | μg | 20 | μg |
| Vitamin B₆ | 2.2 | mg | 2.1 | mg |
| Folic Acid (Vitamin B₉) | 250 | μg | 280 | μg |
| Vitamin B₁₂ | 2.2 | μg | 2.6 | μg |
| Vitamin C | 70 | mg | 95 | mg |
| Vitamin D | 10 | μg | 12 | μg |
| Vitamin E | 10 | mg | 12 | mg |
| Vitamin K | 65 | mg | 65 | mg |
| Iron | 15 | mg | 15 | mg |
| Calcium | 1200 | mg | 1200 | mg |
| Zinc | 19 | mg | 19 | mg |

In fabricating the compositions of the invention, those skilled in the art will recognize that certain excipients may be added to the compositions. Useful excipients include binders (e.g., starch, and gelatin), disintegrants (e.g., starch, clay, and gum), lubricants (e.g., talc and magnesium stearate), diluents (e.g., lactose and starch); colors (GRAS dye); and flavors. The binders are used to help hold the tablet together after compression. Disintegrants are used to help break the tablet apart after ingestion. Lubricants are used to make the powder flow in the tablet machine and to lubricate the steel punches and dies. Diluents are used to add volume to the tablet to increase its size.

The following procedures are exemplary acceptable methods of preparing multi-component vitamin and mineral supplements falling within the scope of the claimed invention. Film coated tablets may be prepared by coating tablets using techniques such as rotating pan coating methods or air suspension methods to deposit a contiguous film layer on a tablet. This procedure is often done to improve the aesthetic appearance of tablets, but may also be done to improve the swallowing of tablets, to mask an obnoxious odor or taste, or to improve to usual properties of an unsightly uncoated tablet. Compressed tablets, for example, without limitation, may be prepared by mixing the components with excipients intended to add binding or disintegration qualities. The mixture is either directly compressed, or granulated then compressed, using methods and machinery quite well known to those in the industry.

In a preferred embodiment, the multi-component vitamin and mineral supplements are fabricated using the methods described in U.S. Pat. Nos. 5,853,760, 6,083,533 and 6,270,798 to Cremer, which are incorporated herein by reference in their entirety. For instance, the multi-component vitamin and mineral supplements may be manufactured by compressing powders, powder mixtures or granular powders by means of conventional tableting tools. Similarly, multi-layer supplements may be produced by compressing each of the layers separately and then combining the layers one on top of the other. Thin matrices may be produced by means of casting or coating methods wherein solutions or suspensions are applied as a thin layer on an intermediate substrate and dried. Additionally, processes using melts, e.g., injection molding and extrusion, are suitable for the production of the supplements of the invention.

Erodible coatings may be manufactured by the same processes as those available for the production of the multi-component vitamin and mineral supplements. It is not important whether multi-component vitamin and mineral supplements and erodible coatings are manufactured one after the other or at the same time. If the adhesion between supplement and erodible coating is insufficient, it may be necessary to use adhesion promoters. These are physiologically acceptable polymers having adhesive properties that continue to exist even in the presence of water. It is also possible that the erodible matrix is an active substance.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

### REFERENCES

1) Hurrell R F, Reddy M, Cook J D. Inhibition of non-haem iron absorption in man by polyphenolic-containing beverages. Br J Nutr. 1999 April;81(4):289-95.
2) Disler P B, Lynch S R, Torrance J D, Sayers M H, Bothwell T H, Charlton R W. The mechanism of the inhibition of iron absorption by tea. S Afr J Med Sci. 1975;40(4):109-16.
3) Gillooly, M., T. H. Bothwell, R. W. Charlton, et al. 1984. Factors affecting the absorption of iron from cereals. Br. J. Nutr. 51:37-46.
4) ABC of Clinical haematology: Iron deficiency anaemia Rebecca Frewin, Andrew Henson, Drew Provan BMJ 1997;314:360
5) Harmening, Denise M., Clinical Hematology and Fundamentals of Hemostasis, Third Edition, F. A., Davis Company, Philadelphia 1997.
6) Stang J, Story M (eds) Guidelines for Adolescent Nutrition Services (2004); Chapter 9
7) Richard L G. Wintrobe's Clinical Hematology. 10th ed. Baltimore: Williams &; Wilkins; 1999:979-1010.
8) Fairbanks V F, Beutler F., Williams Hematology. 6th ed New York: McGraw-Hill: 2001:447-70
9) Hallberg L, Brune M & Rossander L (1986) Effect of ascorbic acid on iron absorption from different types of meal. Human Nutrition: Clinical Nutrition 40,97-113
10) Hallberg L, Brune M. Rossander L (1989) Iron absorption in man: ascorbic acid and dose-dependent inhibition by phytates. Am J Clin Nutr 49:140-144
11) Brune M, Rossander-Hulten L, Hallberg et al (1992) Iron absorption from bread in humans: inhibiting effects of cereal fiber, phytates and inositol phosphates with different numbers of phosphate groups. J Nutr 122: 442-449
12) Hurrell R F, Juillerat M-A, Reddy M B, et al (1992) Soy protein, phytates, and iron absorption in humans. Am J Clin Nutr 56: 573-578.
13) Hallberg L, Brune M, Erlandsson M, Sandber A-S & Rossander-Hulten L (1991) Calcium: effect of different amounts on nonheme- and heme-iron absorption in humans. American Journal of Clinical Nutrition 53, 112-119
14) Whittaker P. Iron and zinc interactions in humans. Am J Clin Nutr 1998;68:442S-446S
15) Hallberg L, Rossander-Hulten L, Brune M, and Gleerup A (1992) Calcium and iron absorption: mechanism of action and nutritional importance. Eur J Clin Nutr 46: 317-327
16) Cook J D, Dassenko S A and Whittaker P(1991) Calcium supplementation: effect on iron absorption American Journal of Clinical Nutrition 53, 106-111
17) Kochanowski B A, McMahan L (1990) Inhibition of iron absorption by calcium in rats and dogs: effects of mineral separation by time and enteric coating. Nutr Res 10:210-226
18) Solomons and Jacob (1981) Studies on the bioavailability of zinc in humans: effect of heme and nonheme iron on the absorption of zinc. Am J Clin Nutr 34:475-482
19) Sandstrom B, Davidsson L, Cederblad A, Lonnerdal B (1985) Oral iron, dietary ligands and zinc absorption. J Nutr 115:411-414
20) Sacks and Houchin, (1978) Comparative bioavailability of elemental iron powders for repair of iron deficiency anemia in rats. Studies of efficacy and toxicity of carbonyl iron. Am. J. Clin. Nutr. 31:566-571
21) Henkin et al., (1991) Niacin revisited: observations on an important but underutilized drug. Am. J. Med. 91: 239-46.
22) Bostom et al., (1996) High-dose B-vitamin treatment of hyperhomocysteinemia in dialysis patients. Kidney Int. 49: 147-52.
23) Robinson et al., (1996) Hyperhomocysteinemia confers an independent increased risk of artherosclerosis in end-stage renal disease and is closely linked to plasma folate and pyridoxine concentrations. Circulation. 94: 2743-48.
24) Locksmith & Duff, (1998) Preventing neural tube defects: the importance of periconceptional folic acid supplements. Obstet. Gynecol. 91: 1027-34.
25) Vanderput et al., (2001) Folate, homocysteine and neural tube defects: an overview. Exp. Biol. Med. 226: 243-70
26) DeFalco et al., (2000) Homocysteinaemia during pregnancy and placental disease. Clin. Exp. Obstet. Gynecol. 27: 188-90.
27) Eskes, (2000) Homocysteine and human reproduction. Clin. Exp. Obstet. Gynecol. 27 157-67

We claim:

1. A multi-component system for delivering a vitamin to a subject comprising:
a) a first layer adapted to release a first component at a first location of the gastrointestinal tract of the subject; and
b) a second layer adapted to release a second component at a second location of said gastrointestinal tract of the subject, said second location being further downstream in said gastrointestinal tract of the subject than the first location,
wherein said first and second components are placed in separate layers to optimize absorption of said components and minimize detrimental interactions between said first component and said second component, and at least one of said first and second components includes a vitamin.

2. The multi-component vitamin delivery system of claim 1, wherein said first component includes at least one vitamin, mineral or trace element that is to be released at said first location.

3. The multi-component system of claim 2, wherein said first component comprises iron.

4. The multi-component system of claim 1, wherein said second component includes at least one vitamin, mineral or trace element that is to be released at said second location.

5. The multi-component system of claim 4, wherein said second component comprises a mineral selected from the group consisting of calcium, zinc, and salts and mixtures thereof.

6. The multi-component system of claim 5, wherein said second component comprises calcium.

7. The multi-component vitamin delivery system of claim 2, wherein said first component comprises a water-soluble vitamin selected from the group consisting of vitamin C, the B-complex vitamins and mixtures thereof.

8. The multi-component vitamin delivery system of claim 4, wherein said second component comprises a fat-soluble vitamin selected from the group consisting of vitamin A, vitamin D, vitamin E, vitamin K and mixtures thereof.

9. The multi-component vitamin delivery system of claim 4, wherein said second component comprises a mineral or trace element selected from the group consisting of chromium, calcium, iodine, zinc, magnesium, molybdenum, selenium, copper and mixtures thereof.

10. The multi-component vitamin delivery system of claim 1, wherein said first location is selected from the group consisting of the stomach and the duodenum.

11. The multi-component vitamin delivery system of claim 1, wherein said second location is selected from the group consisting of the small intestine and large intestine.

12. The multi-component system of claim 1 further comprising at least one additional layer.

13. The multi-component vitamin delivery system of claim 1, wherein said second layer is completely surrounded by said first layer.

14. The multi-component vitamin delivery system of claim 1, wherein said second layer is partially surrounded by said first layer.

15. The multi-component vitamin delivery system of claim 1, wherein said delivery system is in an oral dosage form.

16. The multi-component vitamin delivery system of claim 15, wherein said oral dosage form is selected from the group consisting of immediate release, extended release, pulsed release, delayed release, timed release, variable release, controlled release and combinations thereof.

17. The multi-component vitamin delivery system of claim 15, wherein said oral dosage form is selected from the group consisting of a tablet and a caplet.

18. The multi-component vitamin delivery system of claim 17, wherein said oral dosage form is a tablet selected from the group consisting of a tablet having two or more separable layers and a tablet having two or more layers with at least one layer surrounding the other.

19. The multi-component vitamin delivery system of claim 18, wherein said oral dosage form is a tablet having at least two layers.

20. The multi-component vitamin delivery system of claim 15, wherein said oral dosage form comprises a first coating layer that allows release of said first component from said first layer, said first coating layer selected from the group consisting of a sugar coating, a film coating and an enteric coating.

21. The multi-component vitamin delivery system of claim 20, wherein said oral dosage form comprises a second coating layer that releases said second component from said second layer, said coating layer selected from the group consisting of a microporous membrane and an enteric coating.

22. A method of making a multi-component system for delivering vitamins, minerals or trace elements to a subject comprising:
mixing a first set of components that are to be released at a first location in the gastrointestinal tract of the subject, said first set of components including at least one water-soluble vitamin, mineral or trace element that is to be released at said first location;
forming a first layer that includes said first set of components;
mixing a second set of component that are to be released at a second location in the gastrointestinal tract of the subject, said second location being further downstream in said gastrointestinal tract than said first location, said second set of components including at least one water-soluble vitamin, mineral or trace element that is to be released at said second location;
forming a second layer that includes said second component; and
combining said first layer and said second layer to form said multi-component system, whereby said first and second set of components are selected to optimize absorption and minimize detrimental interactions.

23. The method of claim 22, wherein said first location is located at the stomach or at the duodenum of the subject and said second location is located at the small intestine or at the large intestine of the subject.

24. The method of claim 22 further comprising coating said first layer with a first coating layer that delays the release of the component of said first layer.

25. The method of claim 24, wherein said first coating layer is placed on said first layer by a process selected from spray coating and compression coating.

26. The method of claim 22, further comprising coating said second layer with a second coating layer through which the component of the second layer can diffuse.

27. The method of claim 26, wherein the coating layer provides a lag-time between delivery of the component from the first layer and the subsequent delivery of the component from the second layer.

28. A multi-component vitamin delivery system comprising:
a first layer adopted to release iron in a form absorbable at a first location in the gastrointestinal tract; and
a second layer adopted to release a second set of components at a second location in the gastrointestinal tract, said second location being further downstream in said gastrointestinal tract than said first location, said second set of components includes at least one material that reduces or inhibits the absorption of iron.

29. The multi-component vitamin delivery system of claim 28 wherein said first set of components further includes one or more water-soluble vitamins, minerals and trace elements that are to be released at said first location.

30. The multi-component vitamin delivery system of claim 29 wherein said first set of components includes one or more water-soluble vitamins selected from the group consisting of vitamin C and the B-complex vitamins.

31. The multi-component vitamin delivery system of claim 28 wherein said at least one material that reduces or inhibits the absorption of iron, or whose absorption is inhibited by iron, is selected from the group consisting of calcium, zinc, and salts and mixtures thereof.

32. The multi-component vitamin delivery system of claim 31 wherein said second set of components comprises calcium, and one or more fat-soluble vitamins, minerals and trace elements that are to be released at said second location.

33. The multi-component vitamin delivery system of claim 32 wherein said second set of components includes one or more fat-soluble vitamins selected from the group consisting of vitamins A, D, E and K.

34. The multi-component vitamin delivery system of claim 33 wherein said second set of components include one or more minerals and trace elements selected from the group consisting of chromium, calcium, iodine, zinc, magnesium, molybdenum, selenium and copper.

35. The multi-component vitamin delivery system of claim 28 wherein said first location is selected from the group consisting of the stomach and the duodenum and said second location is selected from the group consisting of the small and large intestines.

36. The multi-component vitamin delivery system of claim 28 wherein said second layer is completely surrounded by said first layer.

37. The multi-component vitamin delivery system of claim 28 wherein said second layer is partially surrounded by said first layer.

38. The multi-component vitamin delivery system of claim 28 wherein said delivery system is in an oral dosage form.

39. The multi-component vitamin delivery system of claim 38 wherein said oral dosage form is selected from the group consisting of immediate release, extended release, pulsed release, delayed release, timed release, variable release, controlled release and combinations thereof.

40. The multi-component vitamin delivery system of claim 38 wherein said oral dosage form comprises a first coating layer that releases said first set of components from said first layer, said first coating layer selected from the group consisting of a sugar coating, a film coating and an enteric coating.

41. The multi-component vitamin delivery system of claim 38 wherein said oral dosage form comprises a second coating layer that releases said second set of components from said second layer, said coating layer selected from the group consisting of a microporous membrane and an enteric coating.

42. The multi-component vitamin delivery system of claim 2 wherein said first set of components includes iron, vitamin C, folic acid and vitamin B12 and said second set of components includes calcium, vitamins A, D, E and K, zinc and copper.
